(19) United States
(12) Patent Application Publication
(10) Pub. No.: US 2006/0159692 A1
(43) Pub. Date: Jul. 20, 2006

(54) Transcriptional factor inducing apoptosis in cancer cell

(76) Inventors: Yoichi Taya, Tokyo (JP); Masato Enari, Tokyo (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/546,829

(22) PCT Filed: Feb. 25, 2004
(86) PCT No.: PCT/JP04/02238

§ 371 Date: Aug. 25, 2005

(30) Foreign Application Priority Data
Feb. 26, 2003 (JP) 2003-04858

(51) Int. Cl. C12N 9/99; A61K 39/00

(52) U.S. Cl. 424/185.1; 530/350; 435/184

(57) ABSTRACT

A new method for treating cancer by inducing apoptosis exclusively in cancer cells and killing them is provided. The present invention relates to a transcriptional factor, comprising p53 or a mutated type p53, wherein one or more amino acids are deleted, substituted or added with respect to the amino sequence of p53, and clathrin heavy chains and having an activity to induce apoptosis of cancer cells. The transcriptional factor enhances the transcriptional activity of p53AIP1 promoter and induces apoptosis of cancer cells.

### TECHNICAL FIELD OF THE INVENTION

The present invention relates to a transcriptional factor inducing apoptosis of cancer cells.

### PRIOR ART

A tumor suppressor gene p53 is a transcription factor which specifically binds to DNA sequences of target genes such as p21, p53R2 or p53AIP1 and controls transcriptional activity of these genes. Therefore the mechanism of cellular tumorigenesis involving p53 has been extensively studied (Cell Technology vol. 22, No. 1, 23-28 (2003)). Among various target genes controlling p53, p53AIP1 is a protein localized to mitochodria, which controls mitochondrial membrane potential and releases cytochrome c, and, therefore, has a function to induce positive apoptotic effect (Oda K. et al., Cell, vol. 102, 849-862, 2000; Matsuda K. et al., Cancer Res., vol. 62, 2883-2889, 2002). The p53AIP1 has a strong correlation to phosphorylation of Ser46, which has an important role in p53-dependent induction of apoptosis (Japanese Patent Application No. 2001-292953), and the expression of p53AIP1 gene induces apoptosis of cancer cells. For example, when cells are exposed to a strong stress such as generation of DNA damage, it is believed that Ser46 of transcriptional factor p53 is phosphorylated by the function of p53DINP1 and so forth, the expression of p53AIP1 is enhanced and apoptosis of cancer cells is induced (Cell Technology vol. 22, No. 1, 23-28 (2003)).

On the other hand, to induce apoptosis of cancer cells, various anticancer drugs such as cisplatin have been used and most of them induce apoptosis by generating DNA damage in cancer cells and in normal cells, which sometimes leads to side effects. Radiation therapy of cancer also induces side effects by generating DNA damage in normal cells.

Furthermore, attempts to induce apoptosis and to kill cancer cells by transfecting cancer cells with adenovirus vectors containing p53 gene, or Bax or p53AIP1, inducible by p53, have been tried but not been successful.

### PROBLEMS TO BE SOLVED BY THE INVENTION

The purpose of the present invention is to provide a new method for cancer treatment by inducing apoptosis exclusively in cancer cells and by killing them.

### MEANS TO SOLVE THE PROBLEMS

The inventors have been studied in solving above-described purpose and found that an about 170 kDa protein, probably attributable to clathrin heavy chains, was co-precipitated with p53 as p53 was expressed in cancer cells and then that the transcriptional activity of p53AIP1 promoter was enhanced when an expression vector carrying cDNA of clathrin heavy chain were transfected into nuclei of cancer cells. Based on these findings, the inventors concluded that the clathrin heavy chains and p53 are associated to compose a transcriptional factor and the transcriptional factor enhances the transcriptional activity of p53AIP1 promoter and induces apoptosis of cancer cells.

That is, the present invention is a transcriptional factor, comprising p53 (SEQ ID NO:1) or a mutated type p53, wherein one or more amino acids are deleted, substituted or added with respect to the amino sequence of p53, and clathrin heavy chains (SEQ ID NO:2) and having an activity to induce apoptosis of cancer cells. These proteins could be extracted and purified from tissues or cells of human and others, or could be obtained from transformants having the DNA coding these proteins.

Moreover, said mutated p53 could be p53, wherein at least the serine residue at amino acid position 46 (hereinafter abbr. as Ser46) is substituted, preferably Ser46 is substituted to Phe.

Furthermore, the present invention is a cancer treatment drug, comprising the transcriptional factor as an effective component.

### BRIEF EXPLANATION OF THE DRAWINGS

FIG. 1 shows a conceptual diagram showing the transcriptional control of p53AIP1 by clathrin heavy chains.

FIG. 2 shows the structure of pc-p53-f vector.

FIG. 3 shows the structure of p53AIP1.pro.reporter vector.

FIG. 4 shows the structure of pc-CHC vector.

FIG. 5 shows the transcriptional activity of p53AIP1 in human osteosarcoma-derived cells, transfected with the expression vector (pcDNA-p53-f).

FIG. 6 shows the SDS-PAGE (silver stained) of the cell lysates from human non-small cell lung cancer-derived cells transfected with the expression vector (pcDNA3.1 or pcDNA-p53-f).

FIG. 7 shows the Western blotting, detected by anti-clathrin heavy chain antibody and anti-p53 antibody, of the cell lysates from human non-small cell lung cancer-derived cells, transfected with the expression vector (pcDNA-p53-f). In the figure, WT, 46A, 46D, 46F and Δ44-63 indicate WT-p53, S46A-p53, S46D-p53, S46F-p53 and 44-63-p53, respectively.

FIG. 8 shows the Western blotting, detected by anti-clathrin heavy chain antibody, of the cytoplasmic and nuclear extracts from human non-small cell lung cancer-derived cells, transfected with the vector (pcDNA3.1, pcDNA-p53-f). In the figure, pcDNA, WT, 46F and Δ44-63 indicate pcDNA3.1, WT-p53, S46F-p53, 44-63-p53, respectively.

FIG. 9 shows the relative transcriptional activation in the cell lysates from human osteosarcoma-derived cells transfected with both the expression vector (pc-CHC) containing cDNA of clathrin heavy chain and the p53 expression vector (pcDNA-p53-f). In the figure, clathrin HC, WT, S46F and Δ44-63 indicate pc-CHC, WT-p53, S46F-p53 and 44-63-p53, respectively.

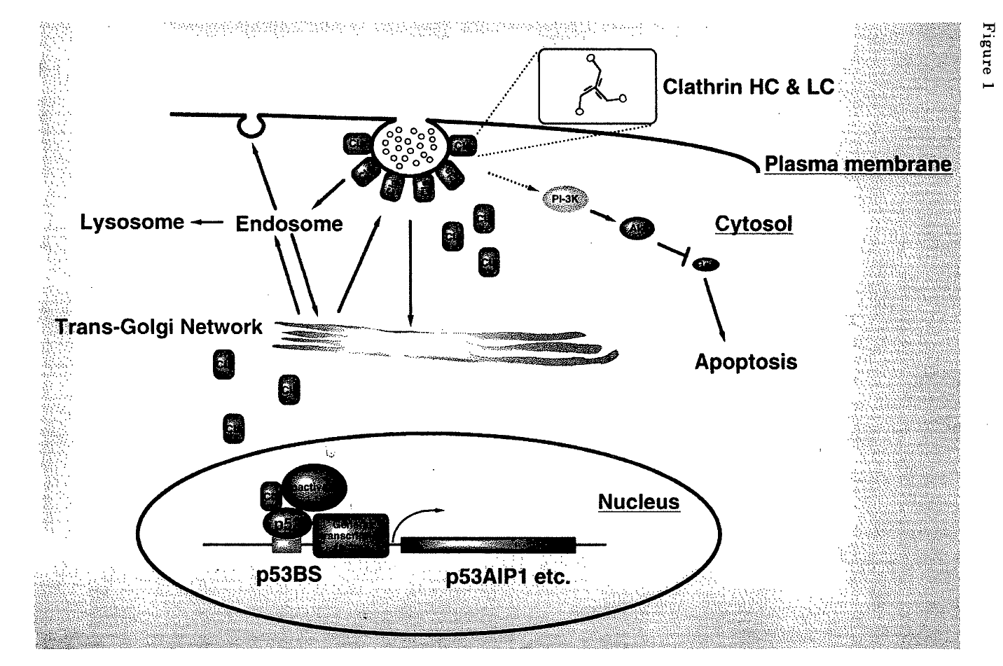

### DETAILED DESCRIPTION OF THE INVENTION

Clathrin has heavy and light chains and, generally, functions for endocytosis, i.e. import of materials from cell surface into cytoplasm. However, the present inventors found that the clathrin heavy chains do not associate with light chains in cancer cells but bind to p53, enhance the transcriptional activity of p53 and induce apoptosis. Clathrin was found to bind strongly to the p53, whose S46 is substituted to phenylalanine, and to induce strong apoptosis.

By injecting the transcriptional factor of the present invention or injecting only clathrin heavy chains into cancer cells by some kind of methods, these bind to p53 inside cancer cells, which induce apoptosis of cancer cells, then kill them.

Using such transcriptional factor would enable specific induction of apoptosis of cancer cells and their death without damaging normal cells more effectively than previous methods. This method, therefore, provides more effective methods for cancer treatment than previous ones.

The following Examples illustrate the invention, however, these are not constructed to limit the scope of the invention.

### TEST EXAMPLE 1

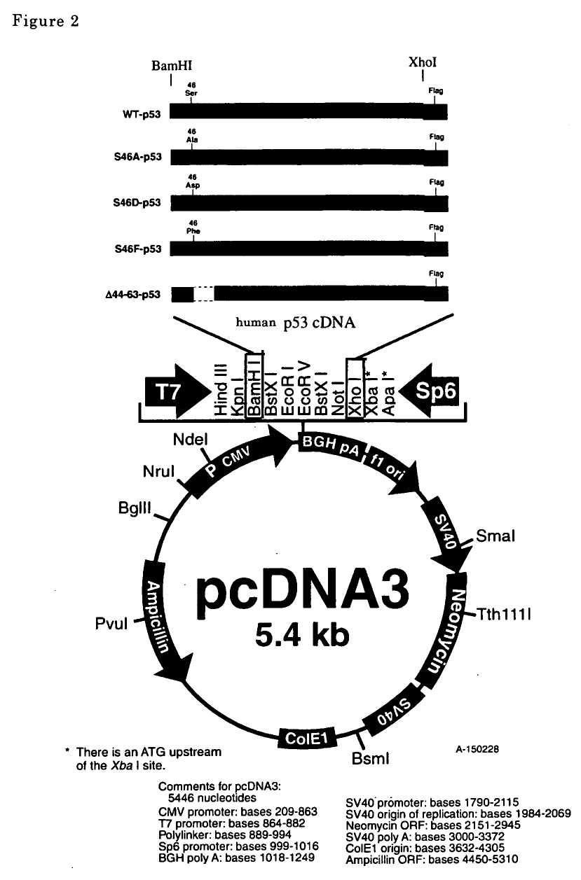

In this Test Example, the inventors prepared the following five kind of vectors (named generically as [pcDNA-p53-f]) used in the subsequent Examples.
(i) A wild type human p53 expression vector (hereinafter referred to as [WT-p53]) comprising wild type human p53 gene inserted in pcDNA3 plasmid (Invitrogen, Ref FIG. 2).
(ii) A mutated type human p53 expression vector (hereinafter referred to as [S46A-p53]) comprising the mutated p53, prepared by the substitution of Ser46 to Ala and inserted to pcDNA3 plasmid.
(iii) A mutated type human p53 expression vector (hereinafter referred to as [S46D-p53]) comprising the mutated p53, prepared by the substitution of Ser46 to Asp and inserted to pcDNA3 plasmid.
(iv) A mutated type human p53 expression vector (hereinafter referred to as [S46F-p53]) comprising the mutated p53, prepared by the substitution of Ser46 to Phe and inserted to pcDNA3 plasmid.
(v) A mutated human type p53 expression vector (hereinafter referred to as [44-63-p53]) comprising the mutated p53, prepared by deletion of amino acid position 44 to 63 and inserted to pcDNA3 plasmid.

The above vectors were prepared according to the following procedures:

For preparation various pcDNA-p53-f, pcDNA3 plasmid (Invitrogen) was cleaved at the restriction sites XhoI and XbaI and inserted with the double-strand DNA (SEQ ID NO: 8) coding a Flag sequence (GDYKDDDDK) comprising of 9 amino acid residues (pcDNA-f). The prepared pcDNA3-f plasmid was cleaved at the restriction sites BamHI and XhoI and was inserted with wild or mutated type human p53 genes (SEQ ID NO:9) with BamHI and XhoI ends (pcDNA-p53-f). The maps of these vectors are shown in FIG. 2.

### TEST EXAMPLE 2

In this Test Example, the inventors prepared a reporter plasmid, wherein about 500 bp DNA containing p53 binding sequence in the intron 1 of p53AIP1 gene was inserted to the upstream of luciferase gene in pGL3-promoter vector (Promega).

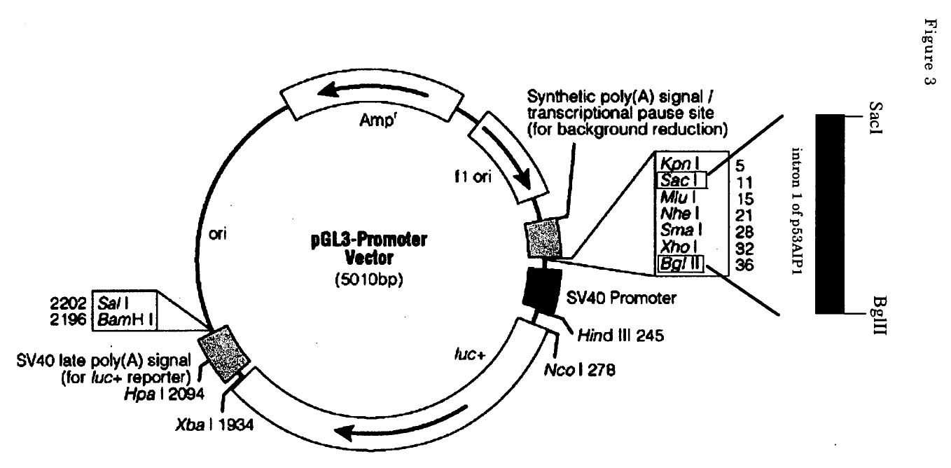

pGL3-promoter vector (Promega) was cleaved at the restriction sites SacI and BglII and was inserted with p53AIP1 intron 1 (about 500 bp, SEQ ID NO: 10) containing a p53 binding sequence with SacI and BglII ends. The map of the vector is shown in FIG. 3. The reporter is hereinafter referred to as [p53AIP1pro. reporter].

### TEST EXAMPLE 3

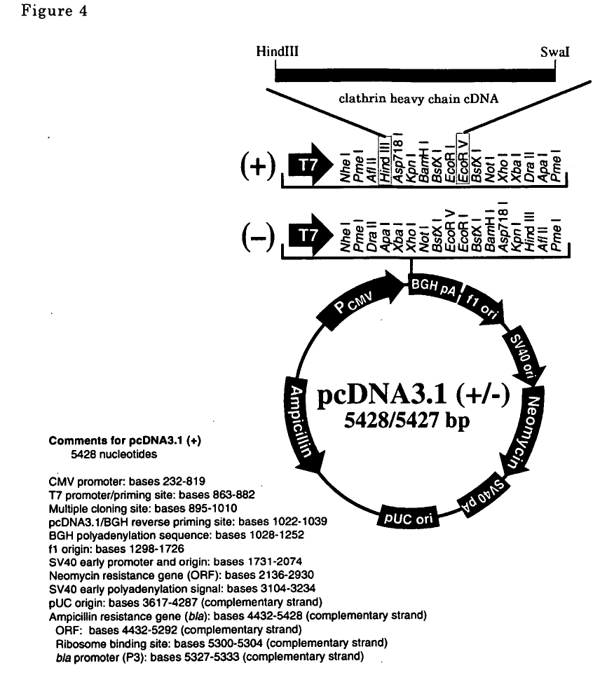

With the similar procedures as Test Example 1, clathrin heavy chain expression vector (hereinafter, referred to as [pc-CHC]) was prepared by insertion of clathrin heavy chain gene (gifted from Kazusa DNA Research Institute, SEQ ID NO: 11) to pcDNA3 plasmid (Invitrogen, Ref. FIG. 2). The map of the vector is shown in FIG. 4.

### TEST EXAMPLE 4

In this Test Example, five kinds of expression vectors prepared in the Test Example 1 were transfected to human osteosarcoma-derived cells and the cell lysate was examined.

The examination was performed by the following procedures.
1) Saos-2 cells (osteosarcoma-derived) were seeded to 24 well tissue culture plate at 7×10⁴ cells/well and cultured for over night.

2) The DNA to be transfected was prepared as shown in Table 1, by the use of pcDNA-p53-f and p53AIP1 pro.reporter prepared in Test Example 1 and 2. Total amount of DNA added was 30 ng with the addition of pcDNA3.1 to 0˜30 ng of each pcDNA-p53-f. In Table 1, phRG-TK shows a plasmid (Promega, Inner control) expressing Renilla reniformis Luciferase. The method for transfection is according to the protocol attached to the kit of Invitrogen.

TABLE 1

| DNA solution | | |
|---|---|---|
| pcDNA3.1 or pcDNA-p53-f | 30 | ng |
| p53AIP1pro.reporter | 100 | ng |
| phRG-TK | 10 | ng |
| Total | 140 | ng |
| OPTI-MEM | 25 | μl |
| Lipo solution | | |
| OPTI-MEM | 25 | μl |
| Lipofectamine 2000 | 0.28 | μl |

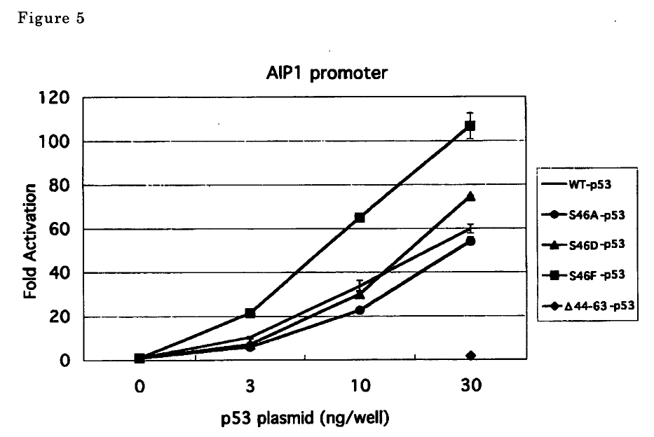

3) DNA solution and Lipo solution were mixed and incubated for 30 min at room temperature.
4) The DNA-liposome complex was dropped to cultures prepared in 1).
5) After 4 hours, DNA-liposome complex was removed and the culture was washed by 1×PBS-.
6) At 24 hours after transfection, the culture was washed by 1×PBS-, and Firefly and Renilla reniformis (the inner control) Luciferase activity was measured by luminometer, respectively. The method for the measurement was according to the protocol of Promega attached to the kit.
7) Relative fold activation was calculated by dividing the activity of Firefly Luciferase by that of Renilla reniformis Luciferase in the lysates from cells transfected with pcDNA 3.1. The results are shown in FIG. 5. Ser46Phe substituent of p53 enhanced the transcription from p53AIP1 promoter much stronger than wild type p53.

### TEST EXAMPLE 5

In this Example, five kinds of expression vectors prepared in Test Example 1 was transfected to human non-small cell lung cancer-derived cells and the cell lysate was examined.

The examination was according to the following procedures.
1) H1299 cells (non-small cell lung cancer-derived) were seeded to 15 cm culture dishes at 9×10⁶/plate (10 plates) and cultured for over night.

2) The DNA to be transfected was prepared according to Table 2. The method of transfection was according to the protocol attached to the kit of Roche Applied Science.

TABLE 2
DNA-liposome solution

| | | |
|---|---|---|
| pcDNA3.1 or pcDNA-p53-f | 8 | μg |
| FuGENE 6 | 48 | μl |
| D-MEM | 752 | μl |

per one dish

3) The DNA-liposome was incubated for 30 min at room temperature and dropped to the cultures prepared in 1).
4) At 21 hours after transfection, cells were split by a scraper and recovered by low speed centrifugation at 600×g for 5 min.

5) The cells were washed once by 1×PBS- and lysed by 10 ml of the cell lysis buffer shown in Table 3.

TABLE 3
Cell lysis buffer

| | | | |
|---|---|---|---|
| 50 | mM Tris-HCl (pH 7.2) | 10 | μg/ml antipain |
| 150 | mM NaCl | 10 | μg/ml pepstatin |
| 2 | mM MgCl₂ | 10 | μg/ml chymostatin |
| 0.1 | mM EDTA | 10 | μg/ml leupeptin |
| 0.1 | mM EGTA | 10 | μg/ml E64 |
| 0.5 | mM DTT | 10 | μg/ml PMSF |
| 1 | mM Na₃VO₄ | 0.1% | NP-40 |
| 10 | mM NaF | | |

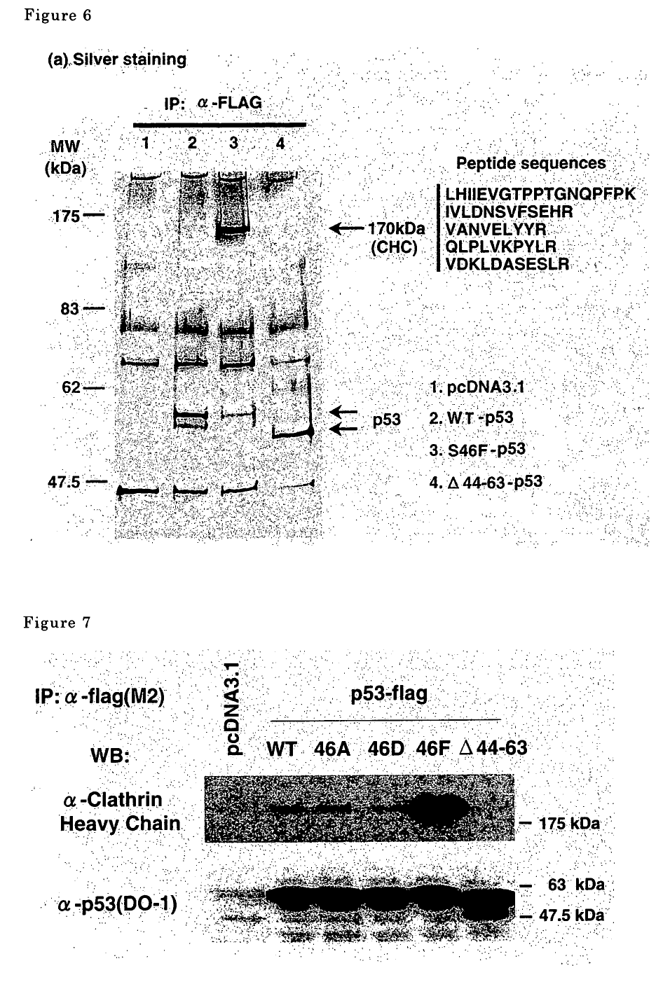

6) After the cells were sonicated, the insoluble fraction was removed by the centrifugation at 15 krpm for 15 min at 4° C.
7) The supernatant was incubated with 0.2 ml of anti-FLAG-anti-agarose beads (Sigma) for over night.
8) After the incubation, beads were washed with the above cell lysis buffer for 4 times.
9) Then, p53 protein was eluted from the beads with the above cell lysis buffer containing 1.5 mg/ml FLAG peptide, competitively.
10) The eluted fraction was loaded on SDS-PAGE and stained with silver. The results were shown in FIG. 6. AS the results, a 170 kDa protein was strongly co-precipitated with p53 in S46F substituent. Analysis by mass spectroscopy of the protein revealed the amino acid sequence of LHIIEVGTPPTGNQPFPK IVLDNSVFSEHR, VANVELYYR, QLPLVKPYLR and VDKLDASESLR (SEQ ID NO: 3˜7). These amino acid sequences correspond to amino acid position 228-245, 1011-1022, 1398-1406, 1444-1453 and 1610-1620 of clathrin heavy chain (SEQ ID NO:2). The results show that the 170 kDa protein is identical to clathrin heavy chain.
11) The identification of the 170 kD protein was performed after the protein was concentrated to 20 times more than the amount used for silver staining, loaded on SDS-PAGE, stained with CBB, cut out the target band, digested by trypsin and analyzed by use of mass spectroscopy.
12) Hundredth part of the eluate obtained in the procedure 10) was performed Western blotting, i.e. the eluate was loaded on SDS-PAGE, transferred to PVDF membrane and stained with anti-clathrin heavy chain antibody (Transduction Laboratories) or with anti-p53 antibody (Santa Cruz). In this Example, the bands were visualized by horseradish peroxidase-conjugated anti-mouse IgG secondary antibody (Amersham) and ECL kit. The results are shown in FIG. 7. Clathrin heavy chains were found to bind strongly to S46F substituent.

### TEST EXAMPLE 6

In this Test Example, nuclear extract from human non-small cell lung cancer-derived cells used in Test Example 5 was examined. The test was according to the following procedures.
1) Cells in a 15 cm culture dish was transfected according to the procedure in Test Example 5.

2) After cell harvest, cells were suspended in 1 ml of the hypotonic buffer* 1 shown in Table 4 and the plasma membrane was disrupted by a homogenizer.

TABLE 4
Hypotonic buffer *1

| | | | |
|---|---|---|---|
| 10 | mM HEPES-KOH (pH 7.9) | 10 | μg/ml antipain |
| 10 | mM KCl | 10 | μg/ml pepstatin |
| 1.5 | mM MgCl₂ | 10 | μg/ml chymostatin |
| 0.5 | mM DTT | 10 | μg/ml leupeptin |
| 1 | mM Na₃VO₄ | 10 | μg/ml E64 |
| 50 | mM NaF | 10 | μg/ml PMSF |

Hypertonic buffer *2

| | | | |
|---|---|---|---|
| 25 | mM HEPES-KOH (pH 7.9) | 10 | μg/ml antipain |
| 420 | mM KCl | 10 | μg/ml pepstatin |
| 10% | glycerol | 10 | μg/ml chymostatin |
| 0.1 | mM DTT | 10 | μg/ml leupeptin |
| 1 | mM Na₃VO₄ | 10 | μg/ml E64 |
| 50 | mM NaF | 10 | μg/ml PMSF |

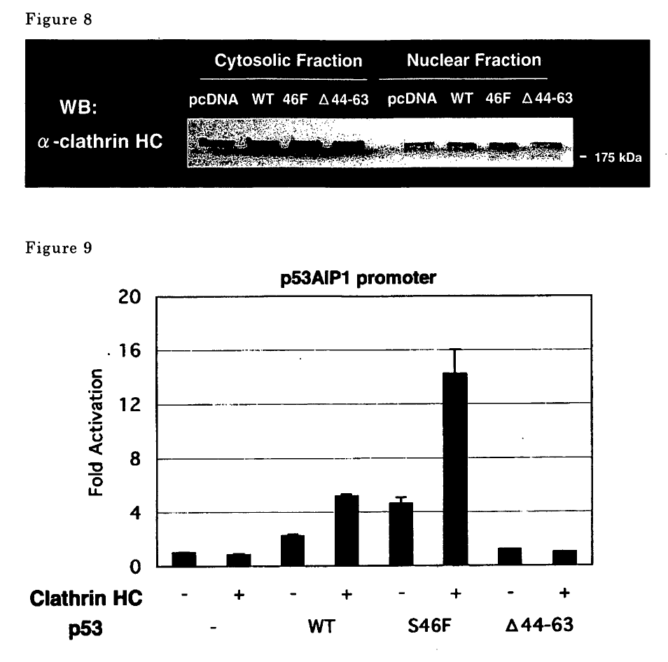

3) The cell debris was centrifuged at 600×g, for 5 min at 4° C. and nuclear fraction was separated from cytoplasmic fraction.
4) Nuclear fraction was sufficiently separated from cytoplasmic proteins by repeating the centrifugation in 2) and 3) for two times.
5) The nuclear fraction was suspended in 0.2 ml of the hypertonic buffer*2 in Table 4, kept on ice for 30 min and proteins were eluted from nuclei.
6) Insoluble fraction was removed from nuclear fraction by centrifugating at 10 k×g for 5 min at 4° C.
7) Proteins from cytoplasmic and nuclear fractions were Western blotted, i.e the proteins were loaded on SDS-PAGE, transferred to PVDF membrane and stained with anti-clathrin heavy chain antibody as described in Test Example 5. The amount of proteins from nuclear fraction was five times more than that of cytoplasmic fraction. The results are shown in FIG. 8. Previously, clathrin was considered to be localized only in cytoplasm, however, a part of clathrin was confirmed to be localized in nuclei.

### EXAMPLE 1

In this Example, experiments were performed according to the procedure in test Example 4 except that the DNA in the DNA solution of 2) was prepared according to Table 5 and used pc-CHC prepared in Test Example 3 as the DNA transfected.

TABLE 5

| DNA solution | | |
|---|---|---|
| pcDNA3.1 or pcDNA-p53-f | 30 | ng |
| p53AIP1pro.reporter | 100 | ng |
| pcDNA3.1 or pc-CHC | 400 | ng |
| phRG-TK | 10 | ng |
| Total | 540 | ng |
| OPTI-MEM | 25 | μl |
| Lipo solution | | |
| OPTI-MEM | 25 | μl |
| Lipofectamine 2000 | 0.28 | μl |

The results are shown in FIG. 9. For the DNA solution, pc-CHC (400 ng) and pcDNA 3.1 (400 ng) solutions are referred to as clathrin + and clathrin −, respectively. Clathrin heavy chain cDNA, inserted to an expression vector and transfected together with p53, was confirmed to enhance the transcriptional activity of p53AIP1 promoter. Especially, the enhancing effect of S46F substituent was predominant.

What is claimed is:

1. A transcriptional factor, comprising p53 or a mutated type p53, wherein one or more amino acids are deleted, substituted or added with respect to the amino sequence of p53, and clathrin heavy chains and having an activity to induce apoptosis of cancer cells.

2. The transcriptional factor of claim 1, wherein at least the serine residue at amino acid position 46 is substituted in said mutated type p53.

3. The transcriptional factor of claim 2, wherein said serine residue at amino acid position 46 is substituted to phenylalanine.

4. A cancer treatment drug comprising the transcriptional factor of any one of claims 1 to 3 as an effective component.

5. A method for treating cancer comprising injecting the transcriptional factor of any one of claims 1 to 3, or clathrin heavy chains to a cancer cell.


SEQUENCE LISTING (11 sequences)

SEQ ID NO: 1 - protein, 393 aa, Homo sapiens [sequence omitted]

SEQ ID NO: 2 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 3 - protein, 18 aa, Homo sapiens
LHIIEVGTPPTGNQPFPK

SEQ ID NO: 4 - protein, 12 aa, Homo sapiens
IVLDNSVFSEHR

SEQ ID NO: 5 - protein, 9 aa, Homo sapiens
VANVELYYR

SEQ ID NO: 6 - protein, 10 aa, Homo sapiens
QLPLVKPYLR

SEQ ID NO: 7 - protein, 11 aa, Homo sapiens
VDKLDASESLR

SEQ ID NO: 8 - protein, 9 aa, artificial sequence
  flag
GDYKDDDDK

SEQ ID NO: 9 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 10 - DNA, 465 bp, Homo sapiens
ccatttcctcagagaacacggcccccttgggccaaaaggacatgaagaagctcttgctaatgccagcctggctctcctcatcccgccccctgcacccctgcccttctggctgccctcccttctcctagctctgtcccctctcacttcaggagtctcaagtccttcagactactccaaagtcgggggatctctggatgggtaggaggtgatctcaccgcctcctctcttgcccgggcttgtcgagatgaacttcctgatgctggcggcgctgaagctgacactagcgggggcacctccctgacatgaacgcccctcgagactgggccagtgctcctgatgcctgggcacctgcggaaaggcacccagcgtggccgccgtggcatgccttgagtgtgtgggtggggactgttgcaaactgacattccagctgtcccagtccattttgtgctgctctcacacaacccc

SEQ ID NO: 11 - DNA, 5028 bp, Homo sapiens
atggcccagattctgccaattcgttttcaggagcatctccagctccagaacctgggtatcaacccagcaaacattggcttcagtaccctgactatggagtctgacaaattcatctgcattagagaaaaagtaggagagcaggcccaggtggtaatcattgatatgaatgacccaagtaatccaattcgaagaccaatttcagcagacagcgccatcatgaatccagctagcaaagtaattgcactgaaagctgggaaaactcttcagatttttaacattgaaatgaaaagtaaaatgaaggctcataccatgactgatgatgtcaccttttggaaatggatctctttgaatacggttgctcttgttacggataatgcagtttatcactggagtatggaaggagagtctcagccagtgaaaatgtttgatcgccattctagccttgcagggtgccagattatcaattaccgtacagatgcaaaacaaaagtggttacttctgactggtatatctgcacagcaaaatcgtgtggtgggagctatgcagctatattctgtagataggaaagtgtctcagcccattgaaggacatgcagctagctttgcacagtttaagatggaaggaaatgcagaagaatcaacgttattttgttttgcagttcggggccaagctggagggaagttacatattattgaagttggcacaccacctacagggaaccagccctttccaaagaaggcagtggatgtcttctttcctccagaagcacaaaatgattttcctgttgcaatgcagatcagtgaaaagcatgatgtggtgttcttgataaccaagtatggttatatccacctctatgatcttgagactggtacctgcatctacatgaatagaatcagtggagaaacaatttttgttactgcacctcatgaagccacagctggaataattggagtaaacagaaagggacaagttctgtcagtgtgtgtggaagaagaaaacataattccttacatcaccaatgttctacaaaatcctgatttggctctgagaatggctgtacgtaataacttagccggtgctgaagaactctttgcccggaaatttaatgctctttttgcccagggaaattactcggaggcagcaaaggtggctgctaatgcaccaaagggaattcttcgtactccagacactatccgtcggttccagagtgtcccagcccagccaggtcaaacttctcctctacttcagtactttggtatccttttggaccagggacagctcaacaaatacgaatccttagagctttgtaggcctgtacttcagcaagggcgaaaacagcttttggagaaatggttaaaagaagataagctggaatgttctgaagaactgggtgatcttgtgaaatctgtggaccctacattggcacttagtgtgtacctaagggctaacgtcccaaataaagtcattcagtgctttgcagaaacaggtcaagtccaaaagattgttttatatgctaaaaaagttggatacactccagattggatatttctgctgagaaatgtaatgcgaatcagtccagatcagggacagcagtttgcccaaatgttagttcaagatgaagagcctcttgctgacatcacacagattgtagatgtctttatggaatacaatctaattcagcagtgtactgcattcttgcttgatgctctgaagaataatcgcccatctgaaggtcctttacagacgcggttacttgagatgaaccttatgcatgcgcctcaagttgcagatgctattctaggcaatcagatgttcacacattatgaccgggctcatattgctcaactgtgtgaaaaggctggcctactgcagcgtgcattagaacatttcactgatttatatgatataaaacgtgcagtggttcacacccatcttcttaaccctgagtggttagtcaactactttggttccttatcagtagaagactccctagaatgtctcagagccatgctgtctgccaacatccgtcagaatctgcagatttgtgttcaggtggcttctaaatatcatgaacaactgtcaactcagtctctgattgaactttttgaatctttcaagagttttgaaggtctcttttattttctgggatccattgttaactttagccaggacccagatgtgcactttaaatatattcaggcagcttgcaagactgggcaaatcaaagaagtagaaagaatctgtagagaaagcaactgctacgatcctgagcgagtcaagaattttcttaaggaagcaaaactaacagatcagctaccacttatcattgtgtgtgatcgatttgactttgtccatgatttggtgctctatttatatagaaataatcttcaaaagtatatagagatatatgtacagaaggtgaatccaagtcgacttcctgtagttattggaggattacttgatgttgactgttctgaagatgtcataaaaaacttgattcttgttgtaagaggtcaattctctactgatgagcttgttgctgaggttgaaaaaagaaacagattgaaactgcttctgccttggctagaggccagaattcatgagggctgtgaggagcctgctactcacaatgccttagccaaaatctacatagacagtaataacaacccggagagatttcttcgtgaaaatccctactatgacagtcgcgttgttggaaagtattgtgagaagagagatccacatctggcctgtgttgcttatgaacgtggccaatgtgatctggaacttattaatgtttgcaatgagaattccctcttcaaaagtctttctcgctacctggtacgtcgaaaggatccagaattgtggggcagcgtgctgctggaaagcaatccttacaggagacccctaattgaccaggttgtacaaacagctttgtctgagactcaggaccctgaagaagtgtcagtaactgtaaaggctttcatgactgcagaccttcctaatgaactcattgaactgctggagaaaattgtccttgataactctgtattcagtgaacacaggaatctgcaaaacctccttatcctcactgcaattaaggctgaccgtacacgtgttatggagtatattaaccgcctggataattatgatgccccagatattgccaatatcgccatcagcaatgagctgtttgaagaagcatttgccattttccggaaatttgatgtcaatacttcagcagttcaggtcttaattgagcatattggaaacttggatcgggcatatgagtttgctgaacgttgcaatgaacctgcggtctggagtcaacttgcaaaagcccagttgcagaaaggaatggtgaaagaagccattgattcttatatcaaagcagatgatccttcctcctacatggaagttgttcaggctgccaatactagtggaaactgggaagaactggtgaagtacttgcagatggcccgtaagaaggctcgagagtcctatgtggagacagaactgatattcgcactggctaaaacaaaccgccttgcagagttagaagaatttatcaatggaccaaataatgctcatatccaacaagttggtgaccgttgttatgatgaaaaaatgtatgatgctgctaagttgttgtacaataatgtttccaattttggacgtttggcatctaccctggttcacctgggtgaatatcaggcagctgttgatggggctaggaaagctaacagtactcgaacatggaaagaggtctgcttcgcctgtgtagatgggaaagaattccgtcttgctcagatgtgtggacttcatattgttgtacatgcagatgaattagaagaacttatcaactactatcaggatcgtggctattttgaagagctgatcaccatgttggaagcagcactgggacttgagcgagctcacatgggaatgtttactgaattagctattctatactctaaatttaagcctcagaaaatgagggagcacctggagctgttctggtctagagtgaatattcccaaggtgctaagagctgcagaacaagctcatctttgggcagaactggtgtttttgtatgacaagtatgaagaatatgataatgccataattaccatgatgaatcatccaactgatgcctggaaagaagggcaattcaaagatatcattaccaaggttgccaatgtggaactatactacagagcaatacagttctacttagaattcaagcctctgttgttaaatgatttgctgatggtgctgtctccacggttggatcacactcgtgcagtcaattatttcagcaaggttaaacagctaccactggtgaaaccgtatttgcgttcagttcagaaccataacaacaaatctgtgaatgaatcattgaacaatctttttattacagaagaagattatcaggctctgcgaacatcaatagatgcttatgacaactttgacaatatctcgcttgctcagcgtttggaaaaacatgaactcattgagttcaggagaattgctgcttatctcttcaaaggcaacaatcgctggaaacagagtgtagagctgtgcaagaaagacagcctttacaaggatgcaatgcagtatgcttctgaatctaaagatactgaattggctgaagaactcctgcagtggtttttgcaggaagaaaaaagagagtgctttggagcttgtctgtttacctgttacgatcttttaaggccagatgtcgtcctagaaactgcatggaggcacaatatcatggattttgccatgccctatttcatccaggtcatgaaggagtacttgacaaaggtggataaattagatgcttcagaatcactgagaaaagaagaagaacaagctacagagacacaacccattgtttatggtcagccccagttgatgctgacagcaggacccagtgttgccgtccctccccaggcaccttttggttatggttataccgcaccaccgtatggacagccacagcctggctttgggtacagcatgtga